(19) United States
(12) Patent Application Publication
(10) Pub. No.: US 2005/0031567 A1
(43) Pub. Date: Feb. 10, 2005

(54) Fixative agents for hair

(76) Inventors: Wilma M. Dausch, Limburgerhof (DE); Bernd de Potzolli, Bad Durkheim (DE); Maximilian Angel, Schifferstadt (DE); Jacqueline Engesser, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/500,138

(22) PCT Filed: Jan. 20, 2003
(86) PCT No.: PCT/EP03/00490

(30) Foreign Application Priority Data
Jan. 21, 2002 (DE) 102 02 253.4

(51) Int. Cl. A61K 7/06; A61K 7/11

(52) U.S. Cl. 424/70.15; 424/70.16

(57) ABSTRACT

Hair-setting compositions comprising A) 0.5 to 20% by weight of a homopolymer of N-vinylcaprolactam or an anionic or nonionic copolymer of at least 70% by weight of N-vinylcaprolactam (polymer A), and B) 0.5 to 20% by weight of a terpolymer (polymer B) obtainable by copolymerization
(a) of at least one C₁-C₄ N-alkylacrylamide or a C₁-C₄ N-alkylmethacrylamide and
(b) at least one C₁-C₄ hydroxyalkyl ester or a C₁-C₄ alkyl ester of acrylic acid or methacrylic acid and (c) acrylic acid and/or methacrylic acid C) 0.5 to 20% by weight of a polymer chosen from the group consisting of copolymers of vinylpyrrolidone and a vinyl ester and of homopolymers of vinylpyrrolidone.

The present invention relates to novel hair-setting compositions which comprise polymer mixtures as film formers.

Hairspray compositions which consist of a polyester and a water-soluble polymer, for example a copolymer of N-vinylcaprolactam and N-vinylpyrrolidone, are known from WO 93/17658.

However, such hairsprays are moisture-sensitive and are therefore of limited use with regard to application and handling.

EP 0 734 717 A2 describes hair-setting compositions comprising
A) 0.5 to 20% by weight of a homopolymer or copolymer of at least 70% by weight of N-vinylcaprolactam (polymer A), and
B) 0.5 to 20% by weight of a further film-forming polymer (polymer B) chosen from the group of polyamides, polyurethanes, homopolymers and copolymers of monoolefinically unsaturated monomers.

An unsatisfactory aspect of the hair-treatment compositions of the prior art is their stickiness.

It is an object of the present invention to develop hair-setting compositions which have good setting properties coupled with good ability to be washed out without exhibiting the above-described moisture sensitivity and which are characterized by low stickiness.

We have found that this object is achieved by hair-setting compositions comprising
A) 0.5 to 20% by weight of a homopolymer of N-vinylcaprolactam or an anionic or nonionic copolymer of at least 70% by weight of N-vinylcaprolactam (polymer A), and
B) 0.5 to 20% by weight of a terpolymer (polymer B) obtainable by copolymerization
   (a) of at least one C₁-C₄ N-alkylacrylamide or a C₁-C₄ N-alkylmethacrylamide and
   (b) at least one C₁-C₄ hydroxyalkyl ester or a C₁-C₄ alkyl ester of acrylic acid or methacrylic acid and
   (c) acrylic acid and/or methacrylic acid
C) 0.5 to 20% by weight of a polymer chosen from the group consisting of copolymers of vinylpyrrolidone and a vinyl ester and of homopolymers of vinylpyrrolidone.

Suitable polymers A are homopolymers of N-vinylcaprolactam which are known to the person skilled in the art and can be prepared, for example, in accordance with the procedure described in U.S. Pat. No. 3,145,147. These polymers are available under the tradename Luviskol® Plus (BASF Aktiengesellschaft).

Further suitable polymers A are copolymers of N-vinylcaprolactam and further polymerizable monomers, where the copolymers consist of at least 70% by weight, preferably at least 85% by weight, of N-vinylcaprolactam.

Suitable further polymerizable monomers for the copolymers A are:
monomers with an acid function, such as acrylic acid, methacrylic acid, acrylamidomethylpropylsulfonic acid (AMPS), 3-sulfopropyl(meth)acrylate, optionally also in completely or partially neutralized form;
C₁-C₁₈ alkyl(meth)acrylates, such as tert-butyl acrylate, ethyl acrylate, isobutyl methacrylate, n-butyl methacrylate, methyl methacrylate, ethyl methacrylate and hydroxyalkyl (meth)acrylates;
vinyl esters of C₂-C₁₀ fatty acids, such as vinyl acetate, vinyl propionate and vinyl esters of longer-chain and/or branched fatty acids, for example versatic acid;
C₃-C₈ N-alkyl(meth)acrylamides, such as methacrylamide, N,N-dimethylacrylamide, N-tert-butylacrylamide and N-tert-octylacrylamide; N-vinylpyrrolidone and N-vinylpiperidone.

The further monomers may be used in the form of an individual compound or in the form of a mixture for the copolymer A.

Such copolymers are known and can be prepared by customary polymerization processes.

For example, EP 455 081 describes copolymers of N-vinylpyrrolidone, N-vinylcaprolactam and N-vinylimidazole. EP 74191 discloses copolymers of N-vinylpyrrolidone, N-vinylcaprolactam and dimethylaminoethyl methacrylate.

For the hair-setting compositions according to the invention, preference is given to using homopolymers of N-vinylcaprolactam as polymers A.

The hair-setting compositions according to the invention comprise the polymers A in an amount of from 0.5 to 20% by weight, preferably from 1 to 10% by weight, based on the finished composition.

Suitable polymers B are terpolymers obtainable by copolymerization
(a) of at least one C₁-C₄ N-alkylacrylamide or a C₁-C₄ N-alkylmethacrylamide and
(b) at least one C₁-C₄ hydroxyalkyl ester or a C₁-C₄ alkyl ester of acrylic acid or methacrylic acid and
(c) acrylic acid and/or methacrylic acid.

In a preferred embodiment, at least 50% of the carboxyl groups present in the terpolymer have been neutralized with a lower organic base chosen from triethanolamine, triisopropanolamine,. 2-amino-2-methylpropanol and 2-amino-2-methyl-1,3-propanediol.

The N-substituted acrylamides or methacrylamides which can be used as monomers (a) are substituted by alkyl radicals which contain 1 to 4 carbon atoms. Examples of such acrylamides and methacrylamides which can be used are N-methylacrylamide, N-ethylacrylamide, N-propylacrylamide, N-n-butylacrylamide, N-isopropylacrylamide, N-sec-butylacrylamide, N-tert-butylacrylamide, and the corresponding methacrylamides. Preference is given to the acrylamides and in particular N-tert-butylacrylamide.

Examples of alkylesters of acrylic acid or methacrylic acid which can be used as monomers (b) are methyl, ethyl, propyl, isopropyl or n-butyl acrylate or methacrylate, preference being given to the acrylates over the methacrylates. In this connection, particular preference is given to ethyl acrylate. Furthermore, acrylates or methacrylates containing hydroxyl groups, such as, for example, hydroxyethyl acrylate, hydroxypropyl acrylate or hydroxyethyl methacrylate, can be used as component (b).

A particularly preferred component (c) is acrylic acid.

Preference is given to polymers B) obtainable from
40 to 60% by weight of (a)
35 to 50% by weight of (b) and
3 to 11% by weight of (c)
   based on the total weight of the monomer mixture—with the proviso that the amounts add up to 100%.

A particularly preferred polymer B) is a terpolymer of acrylic acid/ethyl acrylate/N-tertiary-butylacrylamide. Such copolymers are sold under the tradename Ultrahold® 8, Ultrahold Strong® (BASF Aktiengesellschaft).

Suitable as polymer C) are homopolymers of N-vinylpyrrolidone, as are sold, for example, under the name Luviskol® from BASF Aktiengesellschaft. The copolymers are obtainable by polymerization of N-vinylpyrrolidone with vinyl acetate and/or vinyl propionate in various weight ratios. Examples of such polymers are:
Luviskol® K17, Luviskol® K30, Luviskol® K60, Luviskol® K80, Luviskol® K90 (polyvinylpyrrolidones with a corresponding K value in the form of a powder or in the form of a solution (aqueous or aqueous/alcoholic)).
Luviskol® VA=vinylpyrrolidone/vinyl acetate copolymers, in particular Luviskol® VA 73, Luviskol® VA 64, Luviskol® VA 55, Luviskol® VA 37.

In a particularly preferred embodiment, the hair-setting compositions comprise
A) 0.5 to 20% by weight, in particular 1 to 5% by weight, based on solid, of a homopolymer of N-vinylcaprolactam
B) 0.5 to 20% by weight, in particular 1 to 5% by weight, of a terpolymer of N-tert-butylacrylamide, ethyl acrylate and acrylic acid
C) 0.5 to 20% by weight, in particular 1 to 5% by weight, based on solid, of a homopolymer of N-vinylpyrrolidone and/or a copolymer of N-vinylpyrrolidone and vinyl acetate.

If they are made up of monomers containing acid groups, the polymers A, B and C can be used in the acid form or in partially or completely neutralized form for the hair-setting compositions according to the invention. Suitable agents for the neutralization are alkali metal hydroxides, ammonia, organic amines, in particular aminoalcohols, very particular preference being given to 2-amino-2-methyl-1-propanol.

The polymers A, B and C are present in a solvent, the solvents used preferably being water or alcohols or mixtures of water and lower alcohols. The solvent content in the hair-setting composition is usually 25 to 98% by weight.

The mixing together of the polymers A, B and C can take place by dissolving A, B and C in the form of powders in a solvent, or by adding to a solution of one polymer, the other polymer in each case in the form of a solid or likewise in the form of a liquid.

The hair-setting compositions according to the invention comprise the polymers B and C in an amount of from 0.5 to 20.0% by weight, preferably from 1 to 5% by weight, based on the finished composition.

Besides the polymers A, B and C and a solvent, the hair-setting compositions according to the invention can, depending on the intended use, comprise further customary hair cosmetic additives, such as perfume oils, emulsifiers, preservatives, care substances, such as panthenol, silicone, collagen, vitamins, protein hydrolyzates, stabilizers, pH regulators, dyes, UV filters and further customary additives.

If the hair-setting composition according to the invention is to be used in the form of a hairspray, then a propellant is usually added. Customary propellants are lower alkanes, for example propane or butane, dimethyl ether, nitrogen, nitrous oxide or carbon dioxide or mixtures of these substances. The hair-setting compositions according to the invention can also comprise halogen-containing hydrocarbons as propellant.

In the case of use in mechanical spray devices, for example spray pumps, the propellant can be dispensed with.

The hair-setting compositions according to the invention have excellent performance properties; they form clear films and, in aqueous/alcoholic solutions, have a low solution viscosity, meaning that even in relatively high concentrations they still have good spray properties. Surprisingly, they exhibit a significantly reduced stickiness compared with known hair-setting compositions, in particular consisting of vinylcaprolactam and/or vinylpyrrolidone and/or PVP/VA copolymer.

The polyvinylcaprolactam solutions used in the examples below were 40% strength by weight solutions in ethanol. The Fikentscher K value of the polymer was 40 (1% in ethanol).

### EXAMPLE 1

| | |
|---|---|
| 7.5% by weight | polyvinylcaprolactam solution |
| 2.0% by weight | Ultrahold ® Strong (BASF |
| | Aktiengesellschaft) |
| 1.5% by weight | polyvinylpyrrolidone (PVP) |
| 89.0% by weight | water, dist. |

### EXAMPLE 2

| | |
|---|---|
| 7.5% by weight | polyvinylcaprolactam solution |
| 2.0% by weight | Ultrahold ® Strong (BASF |
| | Aktiengesellschaft) |
| 1.5% by weight | polyvinylpyrrolidone (PVP) |
| 65.0% by weight | ethanol abs. |
| 24.0% by weight | water, dist. |

### EXAMPLE 3

| | |
|---|---|
| 5% by weight | polyvinylcaprolactam solution |
| 2.5% by weight | Ultrahold ® 8 (BASF |
| | Aktiengesellschaft) |
| 2.5% by weight | Luviskol ® K30 (BASF |
| | Aktiengesellschaft): |
| | polyvinylpyrrolidone |
| 90.0% by weight | ethanol abs. |

### EXAMPLE 4

| | |
|---|---|
| 5% by weight | polyvinylcaprolactam solution |
| 2.5% by weight | Ultrahold ® 8 (BASF |
| | Aktiengesellschaft) |
| 2.5% by weight | Luviskol ® K30 (BASF |
| | Aktiengesellschaft): |
| | polyvinylpyrrolidone |
| 90.0% by weight | water, dist. |

### EXAMPLE 5

| | |
|---|---|
| 5.0% by weight | polyvinylcaprolactam solution |
| 2.0% by weight | Ultrahold ® 8 (BASF |
| | Aktiengesellschaft) |
| 2.5% by weight | Luviskol ® VA 64P, |
| | pulverulent copolymer of |
| | N-vinylpyrrolidone/vinyl acetate |
| 52.5% by weight | ethanol abs. |
| 35.0% by weight | water, dist. |

### EXAMPLE 6

| | |
|---|---|
| 5% by weight | polyvinylcaprolactam solution |
| 2.5% by weight | Ultrahold ®, pulverulent terpolymer of |
| | N-tertiary-butylacrylamide, ethyl |
| | acrylate, acrylic acid |
| 1.5% by weight | Luviskol ® K30 (BASF |
| | Aktiengesellschaft): |
| | polyvinylpyrrolidone |
| 0.23% by weight | AMP |
| 53.50% by weight | ethanol abs. |
| 37.27% by weight | water, dist. |

1. A hair-setting composition comprising
A) 0.5 to 20% by weight of a homopolymer of N-vinylcaprolactam or an anionic or nonionic copolymer of at least 70% by weight of N-vinylcaprolactam (polymer A), and
B) 0.5 to 20% by weight of a terpolymer (polymer B) obtainable by copolymerization
(a) of at least one C₁-C₄ N-alkylacrylamide or a C₁-C₄ N-alkylmethacrylamide and
(b) at least one C₁-C₄ hydroxyalkyl ester or a C₁-C₄ alkyl ester of acrylic acid or methacrylic acid and
(c) acrylic acid and/or methacrylic acid
C) 0.5 to 20% by weight of a polymer chosen from the group consisting of copolymers of vinylpyrrolidone and a vinyl ester and of homopolymers of vinylpyrrolidone.

2. The hair-setting composition as claimed in claim 1, wherein a homopolymer of N-vinylcaprolactam is used as polymer A.

3. The hair-setting composition as claimed in claim 1, wherein a terpolymer of N-tert-butylacrylamide, ethyl acrylate and acrylic acid is used as polymer B.

4. The A hair-setting composition as claimed in claim 1, further comprising an alcohol and lower alkanes.

5. The hair-setting composition as claimed in claim 2, further comprising an alcohol and lower alkanes.

6. The hair-setting composition as claimed in claim 3, further comprising an alcohol and lower alkanes.
